(19) United States
(12) Patent Application Publication
(10) Pub. No.: US 2003/0036520 A1
(43) Pub. Date: Feb. 20, 2003

(54) INTESTINAL GENE THERAPY

(76) Inventors: JACK GAULDIE, HAMILTON (CA); BRUCE A. VALLANCE, HAMILTON (CA); STEPHEN M. COLLINS, HAMILTON (CA); YONGHONG WAN, HAMILTON (CA); CHRISTOPHER THOMSON, HAMILTON (CA)

Correspondence Address: VAN DYKE & ASSOCIATES, P.A., 7200 LAKE ELLENOR DRIVE, SUITE 252, ORLANDO, FL, 32809, US

(21) Appl. No.: 09/360,199

(22) Filed: Jul. 23, 1999

(51) Int. Cl. A61K 48/00

(52) U.S. Cl. 514/44

(57) ABSTRACT

In the present invention, a procedure is described which allows for the efficient delivery of gene and cell based vaccines to the rectal mucosal tissue, and which results in efficient and prolonged transferred gene expression in the mucosal tissue resulting in a potent local mucosal immune response directed against the antigen encoded by the administered nucleic acid. This invention provides a unique and potent approach for the development of vaccines and vaccination strategies to develop mucosal immune protective responses in the lower GI and GU tract in the prevention and/or treatment of sexually transmitted diseases and other conditions. This approach also provides a means for the successful transfer of genetic material in gene therapy approaches in the treatment or prevention of colonic diseases.

### FIELD OF THE INVENTION

The present invention provides a method for the effective delivery of biologically active genes to the intestine. Vectors, including adenoviral vectors and other viral vectors or naked DNAs which carry encoded antigen genes or genes encoding biologically active gene products, optionally with cytokine and co-stimulatory molecule genes, are delivered either by themselves, with a pharmaceutically acceptable carrier, or within specific cell types to the rectal mucosa, to induce biologically relevant effects, including elicitation of immune responses within the Gastrointestinal (GI) and Genitourinary (GU) tissues.

### BACKGROUND OF THE INVENTION

The gastrointestinal (GI) tract has many features that make it an attractive site for gene therapy and delivery of other biologically active agents. It offers easy access for the delivery of gene transfer vectors, through both oral and rectal routes. In addition, the entire GI tract is lined by a contiguous layer of epithelial cells, (intestinal epithelial cells, or “IEC's”), to which biologically relevant genes may be presented. Recombinant adenoviruses, the most efficient and extensively used vectors currently available for gene transfer in vivo, can readily infect intestinal epithelial cells (IEC), at least in tissue culture (Jobin, 1998; Cheng 1997). Add to this the potential for modulating the immune system to combat intestinal pathogens (Baca-Estrada, 1995), cancer (Addison, 1995) or inflammation (Jobin 1998; Addison, 1995) and it becomes apparent that intestinal gene therapy should hold great therapeutic promise for both the treatment and prevention of disease. However at present, there have been few studies successfully demonstrating gene transfer to the intestine, and none showing any functional benefits.

Surprisingly, the primary reason for the slow progress in the field has been the lack of an efficient route of intestinal administration of genes and gene transfer vectors. The transfection efficiency obtained by adenoviral vectors through either oral or rectal routes has been very poor, presumably because of interference by the epithelial and mucosal barriers (Sandberg, 1994) which line the gastrointestinal tract, protecting it from noxious and pathogenic agents. Thus, while many advances have recently been made in the development of adenoviral vectors as potential therapeutic tools for a number of applications including intestinal gene therapy (Jobin, 1998; Hogaboam, 1997), testing the efficacy of these vectors has been hampered by the lack of a protocol allowing efficient in vivo transfection of the intestine (Jobin, 1998). As a result, alternative transfection approaches have been developed, involving either surgical manipulation of the intestine (Sferra, 1997; Foreman, 1998), or the systemic administration of adenovectors through the circulation (Brown, 1997). While these approaches have shown some success, the invasiveness and complexity of the surgical procedures and the lack of selectivity for the intestine of systemic vector delivery have prevented either approach from gaining widespread acceptance. The immunisation of the lower GI and GU tract for protection against infectious agents such as viruses, bacteria and mycoplasmas, has proven extremely difficult. Immunisation by introduction of foreign protein (Kleanthous et al 1998) or genes into rectal tissue can be highly effective, but has been technically difficult due to the presence of protective mucus and epithelial barriers. Thus, what is required is a simple, safe approach to intestinal gene transfer, preferably adapting a delivery system already currently used to deliver topical treatments to the GI tract. This invention responds to this long-felt need.

### SUMMARY OF THE INVENTION

According to the method of this invention, genes or gene transfer vectors are efficiently introduced into gastrointestinal cells. Nucleic acids or cells encoding desired functions, or a mixture thereof may all be administered according to the method of this invention. Viruses or plasmids are constructed which contain foreign antigen genes, or genes encoding biologically or clinically relevant gene products, alone or in combination with cytokine or co-stimulatory molecules, genes, chemotactic molecules or genes or angiostatic molecules or genes. The vectors are used by themselves (“nucleic acid-based vaccines”) or after they have been introduced into Dendritic cells and administered to the host as a population of living cells (“cell-based vaccines”). The colonic mucosa is pre-treated or simultaneously treated to cause a breach, preferably temporarily, in the intestinal protective lining to facilitate nucleic acids, gene vectors, recombinant virus vectors, or recombinant cells to contact cells of the mucosa and submucosa, resulting in infection or transfer of the biologically active nucleic acids into the intestinal epithelial and other cells or penetration of the mucosal tissue by cell-based vaccines.

In one embodiment, this invention provides a process whereby the colon is pre-treated with an intrarectal enema of 50% ethanol or like non-toxic mucosal barrier disruptive agent, followed by intrarectal administration of adenovirus or other nucleic acid vector encoding a gene encoding a tumour antigen (PymT antigen, Wan et al 1997). This results in efficient gene transfer to colonic epithelial cells, M cells or both, and antigen expression, which in turn results in Cytotoxic T cell (CTL) generation in the draining ileac lymph node specific for the PymT antigen.

Therefore, it is an object of the present invention to provide a highly efficient, reliable, and simple method for immunisation of the colonic mucosa and to provide specific immune protection at the GI and GU mucosa.

Another object of this invention is to provide a method for delivery of genes, nucleic acid vectors or cells encoding foreign genes or gene vectors to the intestinal epithelial and other cells.

Further objects of this invention will become apparent from a review of the complete disclosure and the claims appended hereto.

### BRIEF DESCRIPTION OF DRAWINGS

FIG. 1. Histological demonstration of beta-galactosidase expression. Panel A. High power macroscopic lumenal view of distal colon that received AdLacZ virus by enema 1 day previous without ethanol pretreatment and stained for beta-galactosidase activity (×50). Note that few positive cells are seen. Panel B. Macroscopic view of distal colon stained as for (A), but the virus was delivered 3 hours after ethanol pretreatment of the colon (×50). Note the impressive increase in the number of blue stained cells. Panel C. Macroscopic view of proximal colon of mouse treated as in B, pinned onto petri dish. Note the strongly infected dome shaped areas (arrow). These have been putatively identified as colonic M cells. Panel D. Microscopic appearance of cross-sectioned colonic mucosa removed from a mouse infected one day previous with LacZ virus and subsequently stained for β-Gal activity. Note the numerous blue stained epithelial cells on the mucosal surface (×100). Panel E. Higher magnification of colonic mucosa 3 days after infection with AdLacZ virus, with numerous epithelial cells still positive for β-Gal (×400). Note that epithelial cells positive for the β-Gal enzyme were reduced in number after day 3 PI. Panel F. While epithelial cells are the predominant cell type infected, occasional cells in the lamina propria were also positively stained, see arrow (×100).

FIG. 2. Luciferase Expression and Distribution

Panel A: Luciferase enzyme activity was assessed in various tissues one day after infection, and is expressed in relative light units (RLU)/mg of tissue. Results are the mean ±1 SEM of groups of 4-6 animals. The asterisk denotes luciferase activity significantly elevated over background. Note that background activity was <1 RLU/mg tissue and no luciferase expression was detected in the spleen, liver, mesentery or iliac lymph nodes following enema delivery of the AdLuc virus. Panel B: Luciferase enzyme activity was assessed in the distal colon over an 8 day time course and is expressed in relative light units (RLU)/mg of tissue. Results are the mean ±1 SEM of groups of 4-6 animals. The asterisk denotes luciferase activity significantly elevated over background. Note that background activity was <1 RLU/mg tissue.

FIG. 3. Immune Response

Induction of PymT-specific lytic activity by lymphocytes from mice vaccinated with AdPymT delivered intrarectally (top panel) and intradermally (bottom panel). Lymphocytes were harvested from mice 5 days after vaccination and tested for cytolytic activity in a ⁵¹Cr-release assay using 516MT3 and control PTO516 cells as targets. Effector cells only lysed PymT-expressing cells. These data are representative of two experiments performed.

FIG. 4 illustrates the induction of antigen specific CTL after administration of dendritic cells transfected with Adenovirus vector encoding gp100 tumour antigen gene from melanoma. The DC were administered subcutaneously and the spleen cells were isolated 14 days after administration of the Cell based vaccine. Induction of gp100-specific lytic activity by splenocytes from C57BL/6 mice vaccinated with DCs transduced with Adgp100. Splenocytes were harvested from mice 14 days after vaccination with DCAdgp100 or Adgp100 alone. Five days after restimulation with target cells BB16F10, effectors were harvested and tested for cytolytic activity in a ⁵¹Cr-release assay using B16F10 (A) and control EL4 (B) target cells. Data presented are representative of five independent experiments.

### DETAILED DESCRIPTION OF THE PREFERRED EMBODIMENTS

This invention provides a direct and clinically applicable approach to intestinal gene therapy, comprising delivery of adenoviral or other gene vectors to the colon by intra-rectal enema, suppository, lavage, instillation or the like. We have discovered that pretreatment with a mucosal barrier breaker facilitates transduction of the colonic epithelium. We have found that pretreatment or concurrent treatment with ethanol or other mucolytic or muco-disruptive agents strongly enhances transfection of the colonic mucosa. Expression of reporter transgenes delivered to the colon following or concurrent with such treatment has allowed us to identify the cell types infected or transefected, as well as the duration and tissue selectivity of the expression. Also demonstrated herein is the application of colonic gene transfer to achieve local immunization, through the expression of adenovector or other gene vector encoded tumor antigens, as a treatment or prophylactic method for tumors. This approach generated a strong cytotoxic T lymphocyte (CTL) response, targeting the transgenic antigen, in the lymph nodes draining the colon.

These results not only demonstrate that intestinal gene transfer is feasible following a reduction in the barriers to transfection or infection, but also indicate that gene transfer to the colon is useful as a route for local immunization, as well as for other intestinal gene therapy applications to prevent or treat intestinal or systemic diseases.

According to the method of this invention, we have found that pre- or concurrent treatment of the colon with agents that breach the mucosal barrier can allow penetration of adenovirus and other viral or non-viral gene vectors to transfer genes to the colonic mucosa and initiate immune responses or other biologically or clinically relevant responses in the local draining lymph nodes of the GI and GU tracts. In addition, we have found that introduction of antigens as adenovirus encoded genes into dendritic cells prior to intra-rectal administration of such cells is a highly effective way of inducing specific cytotoxic cell activity against tumours (see, for comparison, Wan et al 1999, who demonstrated efficient induction of anti-tumor activity through dendritic cell presentation of tumor antigens through routes of administration other than intestinal).

One embodiment of the present invention provides a method of treating the colonic tissue so as to make it accessible for infection of the colonic epithelial cells and for efficient gene and cell transfer to the colonic mucosal tissue. Treatment with a 50% ethanol solution delivered as an enema disrupts the film of mucin covering the colonic mucosa and exposes the colonic epithelium. Administration of adenovirus vectors (5×10⁸ pfu) in physiologic fluids to the treated rectal tissue causes infection to occur and extended gene expression from infected colonic epithelial and other cells (at least 8 days) inducing potent CTL antigen specific responses in the lymph node draining the lower GI and GU tissues. This local stimulation of immunity avoids the difficulties introduced by other upper gastric routes of administration of antigen or gene vectors, namely the possible introduction of tolerance through the oral route.

Adenoviruses (Ads) can be used as mammalian cell expression vectors, with excellent potential as live recombinant viral vaccines, as transducing vectors for gene therapy, for research, and for production of proteins in mammalian cells. As is known in the art, the construction of adenovirus vectors can be performed in many ways. One of the most frequently used and most popular methods for construction of adenovirus vectors is based on “the two plasmid method” whereby suitable host cells (typically 293 cells) are cotransfected with two plasmids, each of which separately is incapable of generating infectious virus, but which, when recombined within the transfected cell can generate replicating virus. The most widely used plasmids of this type are described in PCT publication number WO95/00655, hereby incorporated by reference. This system has advantages over other methods using viruses or viral DNA as components since only easily prepared plasmid DNAs are needed, and there is little or no background of parental virus contamination of the final vector isolates. Furthermore, the plasmids are not only easy and inexpensive to produce by those skilled in the art, but can be easily stored and transported, making them convenient for commercial distribution, (i.e. particularly when precipitated with ethanol or when lyophilized, these vectors do not require a cold chain for distribution). The vectors can be administered directly by injection, instillation, suppository, lavage, bolus or like means, or as a cell-based vaccine, whereby the adenovirus vector encoding an antigen gene is first introduced into antigen presenting cells such as Dendritic cells prior to administration of the infected cells to the host in the form of a bolus, suppository, instillation, lavage or like means.

In the human Ad genome, early region 1 (E1), E3, and a site upstream of E4 have been utilised as sites for introducing foreign DNA sequences to generate adenovirus recombinants. In the absence of compensating deletions in E1 or E3, a maximum of about 2-kb can be inserted into the Ad genome to generate viable virus progeny. The E1 region is not required for viral replication in complementing 293 cells, or other cells known to complement E1, and up to 3.2 kb can be deleted in this region to generate conditional helper independent vectors with a capacity of 5.0-5.2 kb. In the E3 region, which is not required for viral replication in cultured cells, deletions of various sizes have been utilised to generate non-conditional helper independent vectors with a capacity of up to 4.5-4.7 kb. The combination of deletions in E1 and E3 permits the construction and propagation of adenovirus vectors with a capacity for insertions of up to approximately 8 kb of foreign DNA.

It will be appreciated that other vectors and/or gene formulations such as plasmid DNA can be administered to the rectal tissue in a similar manner. In addition, it will be appreciated that dendritic cells can be administered by this route to colonic mucosal tissue and affect the local (ileac lymph node) mucosal lymphoid tissue.

It will be appreciated that other solutions and/or treatments can be used to treat the mucosal barrier to allow introduction of the antigen genes. Thus, any non-toxic agent which causes partial, and preferably temporary, disruption of the mucosal barrier may be used to pre-treat or concurrently treat the intestinal lining, to enhance gene delivery to the intestinal epithelial and other cells. Different concentrations of ethanol, alone or in combination with other agents may be used to achieve this result. Thus, an ethanol concentration of between about 5% and 75%, or preferably 25-60% and most preferably, about a 50% ethanol solution is contacted with the intestinal lining. Other alcohols, including but not limited to propanol, methanol, and the like may be used in a similar fashion, so long as toxic effects, including permanent disruption of the intestinal mucosal lining, does not result. Other agents that may be used effectively include mucolytic agents, such as mucolytic enzymes, N-acetyl cysteine, or penetration enhancing agents, such as DMSO, may likewise be included in compositions for gene transfer to the intestinal cells, either alone or in combination with ethanol. It will be appreciated that if various nucleic acid constructs are contacted with the intestinal lining in a precipitated state, as in plasmids precipitated in ethanol, as the ethanol concentration drops, the nucleic acids become solubilized and are taken up by the intestinal cells. In this manner, the time course of gene expression and transfer may be modulated to achieve longer or shorter gene expression time courses. In addition, as necessary, the treatment may be repeated to achieve long-term treatment objectives. Furthermore, the nucleic acid constructs thus presented may be monospecific (i.e. encoding one active gene, or may be multispecific, encoding multiple gene products, antisense gene products and the like, and may even be mixtures of different biologically active gene constructs).

The treatment can be administered at a prior time to vector administration or it can be administered simultaneously with the nucleic acid or nucleic acid vector such as in a combined suppository preparation. In one embodiment of this invention, we have found that pre-treatment of the intestinal lining with an ethanol composition followed by a delay of several hours optimises the level of gene expression upon subsequent contact of the thus-treated intestine with genetic material. However, those skilled in the art will appreciate that by appropriate manipulation of the treating agent, the time course for delivery of nucleic acid may be modified. Thus, for example, presentation of nucleic acid compositions simultaneously with DMSO is preferred to separating the time course of DMSO treatment and the nucleic acid presentation step.

It will be appreciated that the term “bacterial plasmid” is not meant to be limiting, since one skilled in the art would recognise that other types of DNA could be used to achieve antigen expression with equal efficiency. For example, adenovirus vector systems may be used to allow for extended expression, such as those described for a “helper-dependent” adenoviral vectors. Expression of known antigen genes includes, but is not limited to genes encoding tumour antigens, viral and bacterial antigens and mycoplasma antigens. This method of administration could also be used to deliver other functional genes to the rectal mucosa, such as anti-inflammatory genes, tissue matrix stimulating genes and genes to modify local autoimmune responses.

It will be appreciated by those skilled in the art that the present invention disclosure provides significant advances over techniques known in the art for generation of local GI and GU mucosal immunity. It will also be appreciated that while the present disclosure refers throughout to treatment of the intestinal tract with a mucous membrane disruptive or mucolytic agent, such treatment and method may equally be applied to treatment of the genitourinary tract, and the claims appended hereto should be so interpreted. However, since it is considered likely that such treatment would be met with significant resistance in practice, treatment of the gastrointestinal tract is focused on herein as the principal application to which the instant method is applied. First, the efficiency by which the gene is expressed within the colonic mucosa for an extended period of time enhances the extent and strength of the mucosal response. Second, the route of administration avoids the possible introduction of oral tolerance, common to other mucosal routes of administration.

In reviewing the detailed disclosure which follows, it should be borne in mind that any publications referenced herein are hereby incorporated by reference in this application in order to more fully describe the state of the art to which the present invention pertains.

While Wirtz et al, 1999, disclosed intra-rectal administration of a recombinant adenovirus encoding foreign genes in mice two days after induction of experimental colitis (inflamed colon and severe diarrhea, weight loss, and rectal prolapse, resembling Crohn's disease in humans, see Neurath et al., 1995) using trinitrobenzenesulphonic acid as an irritant, and Shibata et al (1997) were able to induce adenomas in mouse rectums via injection of Cre recombinase encoding adenovirus into the colorectum of starved mice, the present invention provides the first demonstration of rapid and efficient adenoviral vector-mediated antigen gene transfer to the normal colon by enema delivery using a mucus membrane disruptive agent which does not induce pathology. The present invention therefore confirms and extends previous reports indicating that IEC can be infected by recombinant adenovectors, both in vitro (Jobin et al., 1998; Cheng et al., 1997) and in vivo (Sferra, et al., 1997; Foreman et al., 1998; Brown et al., 1997). While this invention disclosure focuses on the description of a novel and highly efficient protocol to transduce the colonic epithelium with adenoviral vectors, those skilled in the art will appreciate that other vectors may be used in a similar fashion.

Without wishing to be bound by theoretical considerations, it is considered likely that the poor transfection efficiency heretofore experienced by those skilled in the art was due to the colonic mucus barrier preventing vectors from reaching the epithelium. This invention provides a method for transient disruption of the mucus layer to achieve efficient transfection. Once the impeding mucus layer was circumvented, administration of adenovirus to the lumen of the colon resulted primarily in the transduction of epithelial cells. It is further apparent from the results disclosed herein that proximal colonic M cells were also infected by this approach. Relatively sparse transduction of cells in the lamina propria and intestinal crypts, suggests that the colonic epithelium remained intact and protected the cells in these regions from exposure to the virus. This finding agrees with our observations that the ethanol pretreatment had a minimal impact on the histological appearance of the colon, with infected tissues maintaining an intact epithelial layer and showing little evidence of inflammation beyond that attributable to the normal host response to adenoviral infection.

As the primary cell type transfected by this approach, colonic enterocytes are well positioned to serve as target cells for intestinal gene therapy. Their proximity to mucosal immune cells and their ability to present antigen has led to their consideration as active participants in the mucosal immune system, and important contributors to immune regulation within the gut (Mayer, 1997). Moreover, a recent study infecting epithelial cells with viral vectors in vitro found that two thirds of the transgenic protein was secreted across the epithelium in a basolateral direction (Lozier et al., 1997). Based on the strong immune response we have been able to generate against the PymT antigen by lymphocytes found in the iliac nodes, we have demonstrated that using the methods disclosed herein, this desirable result can be made to occur in vivo.

One potential drawback to the transfection of enterocytes is their relatively rapid turnover, with the process of enterocyte proliferation, differentiation, and migration to the apical extrusion zone being complete over the course of 3-4 days in the colon (Lipkin et al., 1963). In fact, it was recently stated that the biggest remaining challenges to overcome in the development of intestinal gene therapy are the natural antiviral defense provided by the mucus barrier and the rapid turnover of intestinal epithelial cells (Jobin et al., 1998). According to the method of the present invention, the first challenge has been overcome by circumventing the mucus barrier. The second challenge of epithelial cell turnover remains. Indeed strong expression of reporter genes as disclosed herein was observed for 2-3 days, likely reflecting the turnover and loss of the transduced enterocytes. As for the limited luciferase expression seen until day 8 PI, this likely reflects the small number of transduced cells found in the lamina propria or within the crypts. In addition, since adenoviral vectors can induce an anti-viral host response (Yei et al, 1994), and do not usually integrate into their hosts DNA, such vectors are typically capable of providing only transient transgene expression no matter what organ or cell type they infect. Accordingly, those skilled in the art will appreciate that if longer-term gene expression is desired than can readily be achieved using adenoviral vectors, other gene transfer vectors, including retroviruses, may be employed to achieve transgene integration into host genomes. Furthermore, as disclosed above, even using adenoviral vector constructs, by presenting the nucleic acids in a bolus, suppository, precipitated or complexed form, a time-release effect may be achieved, thus extending the period of transgene presentation and expression may be extended, regardless of enterocyte turnover.

Based on their ability to generate strong but transient transgene expression, adenovectors have shown promise as vectors capable of immunizing against rabies and herpes viruses and more recently cancer, through DNA vaccination (Rolph, 1997). The concept is based on the identification of specific immunogenic antigens and the genes encoding them, which are then expressed by adenoviral vectors. Thus immunity against a number of viral and bacterial pathogens, as well as various forms of cancer can be raised without using the original organisms or cells. This concept is particularly attractive when considering that a single multipurpose vector could be used to generate immunity against a wide variety of antigens simply by changing the encoded transgene. Such vaccination would have wide application within the GI tract, with the increasing prevalence of colon cancer (Parkin et al, 1999) and the growing risk of infection by antibiotic resistant strains of bacteria (French, 1998) and other pathogens. This invention disclosure indicates that for any of these applications, vaccination against tumor antigens, production of biologically relevant gene products, and the like, even the transient and presumably local expression of the PymT antigen, was sufficient to induce a strong cytotoxic response in the draining iliac lymph nodes, capable of lysing target cells almost as effectively as cells immunized by intradermal injection of the vector. We chose to test the response to PymT antigen, as a transgenic model of PymT induced cancer in mice has been well characterized, particularly with respect to the ability of AdPymT to vaccinate against tumorigenesis (Wan et al., 1997). In fact intradermal vaccination using AdPymT has already been shown to induce a specific immune response capable of preventing the formation of breast tumors in this model (Wan et al., 1997), and work is currently underway to adapt the model to produce and demonstrate prevention of tumors in the colon. Thus, the method disclosed and claimed herein, namely intrarectal immunization, is considered to be likely to prove to be the most effective way to develop local immunity against colonic tumor formation.

The release of transgenic proteins by epithelial cells into the lamina propria should have significant potential for affecting intestinal immune responses, not only through the delivery of DNA vaccines but also through the local release of immunomodulatory agents. While most research in gene therapy has been directed towards permanent gene replacement, as a cure for genetic diseases (Wilson, 1995), this has few applications in the GI tract, since specific monogenic defects have not been identified in chronic GI conditions. Therefore, for most intestinal diseases, an approach mimicking that taken with conventional treatments, where treatment is usually given only when the disease is overt, would be more useful. Thus another area of interest for intestinal gene transfer would be the use of IECS (intestinal epithelial cells) as sites for the manufacture and secretion of peptides and proteins involved in the treatment of inflammatory bowel disease (IBD). Immunotherapy as a means to treat chronic IBD has come of age in the last few years, (MacDonald, 1998) and both experimental and clinical trials have shown some success. In fact, it has recently been demonstrated that interleukin (IL)-4 (Hogaboam et al, 1997) and IL-10 expressing adenovectors, delivered intra-peritoneally were protective during experimental colitis in the rat. Unfortunately, such anti-inflammatory therapies were not selectively targeted to the intestine. Gene therapy delivered by enema, suppository, bolus, instillation, or like means to the colon would offer the advantage of tissue selectivity, since as disclosed herein, no transgene expression was detected outside of the colon when using the method of this invention. Using this approach, infected IECs are used to locally produce and secrete immunosuppressive proteins such as IL-10, IL-1 receptor antagonist and TGF-β, as well as growth factors to increase re-epithelialization of diseased tissues. Such localized transgene expression, especially over a period of a few days, should prove safer, more effective, and more physiological than alternative systemic approaches such as the intravenous injection of recombinant proteins that rapidly disappear from the serum.

Thus, the protocol disclosed herein allows for the efficient transduction of colonic epithelium with adenoviral vectors, retroviral vectors or naked biologically active gene constructs capable of expressing antisense or sense gene products through topical administration to the colon. This approach offers the added benefit of generating transgene expression selectively in the colon, and is the first study to show the feasibility and efficiency of immunization or gene therapy through DNA vaccines or therapeutics, as one potential application of gene transfer targeting the colon. Furthermore, this protocol allows for the testing of potential genetic therapies in a number of in vivo models of intestinal diseases, and provides significant information concerning the effects of transient transgene expression on colonic physiology and both systemic and mucosal immunity. While the routine use of genetic therapies in the treatment of GI diseases is still in the future, the present demonstration that the colonic epithelium is amenable to transfection prompts the testing of mucolytic agents and other clinically applicable treatments to facilitate gene transfer to the human and animal colon. Such methods should be considered to come within the scope of the instant invention and the claims appended hereto.

Having generally described the purposes, advantages, applications and methodology of this invention, the following specific examples are provided to describe in a detailed fashion, various embodiments of this invention. However, it should be appreciated that the invention described herein is not limited to the specifics of the following examples, which are provided merely as a guide for those wishing to practice this invention. The scope of the invention is to be evaluated with reference to the complete disclosure and the claims appended hereto.

The following examples using the human adenovirus serotype 5 are not meant to be limiting. One skilled in the art would realise that similar plasmids, viruses and techniques could be utilised with different adenoviral serotypes, for example Ad2. Similarly, the use of human Ads is not meant to be limiting since similar plasmids, viruses and techniques could be utilised for different non-human adenoviruses, for example bovine. Similarly, the use of adenoviruses is not meant to be limiting since similar plasmids, viruses and techniques could be utilised with other viruses, both human and non-human, for example baculovirus, or nonviral nucleic acid constructs may be used, as in naked DNA or RNA gene delivery methods known in the art.

It is important to an understanding of the present invention and the examples that follow to note that all technical and scientific terms used herein, unless otherwise defined, are intended to have the same meaning as commonly understood by one of ordinary skill in the art. The techniques employed herein are also those that are known to one of ordinary skill in the art, unless stated otherwise.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

The terms used herein are not intended to be limiting of the invention. For example, the term “gene” includes cDNAs, RNA, or other polynucleotides that encode gene products. “Foreign gene” denotes a gene that has been obtained from an organism or cell type other than the organism or cell type in which it is expressed; it also refers to a gene from the same organism that has been translocated from its normal situs in the genome. In using the terms “nucleic acid”, “RNA”, “DNA”, etc., we do not mean to limit the chemical structures that can be used in particular steps. For example, it is well known to those skilled in the art that RNA can generally be substituted for DNA, and as such, the use of the term “DNA” should be read to include this substitution. In addition, it is known that a variety of nucleic acid analogues and derivatives are also within the scope of the present invention. “Expression” of a gene or nucleic acid encompasses not only cellular gene expression, but also the transcription and translation of nucleic acid(s) in cloning systems and in any other context. The term “gene product” refers primarily to proteins, polypeptides, and antisense genes encoded by nucleic acids (e.g., non-coding and regulatory RNAs such as tRNA, sRNPs, mRNAs, cDNAs, genomic DNA and the like). The term “regulation of expression” refers to events or molecules that increase or decrease the synthesis, degradation, availability or activity of a given gene product. The term “biologically active” as it is used in connection with nucleic acid constructs means that a gene, the expression of which is desired, is under the regulatory control of appropriate transcription initiation and termination factors, and that all needed translation start and stop signals are provided for. The term immune response refers to both cellular and humoral immunity and includes all T cell subtypes and all class of immunoglobulins. The term mucosal immune response refers to the normally occurring and induced immune response at or in the mucosal tissue, including, but not restricted to nasal, bronchial and lung tissue, stomach, intestine, colon, and genitourinary tract.

The present invention is also not limited to the use of the cell types and cell lines used herein. Cells from different tissues (breast epithelium, colon, lymphocytes, etc.) or different species (human, mouse, etc.) are also useful in the present invention.

It is important in this invention to detect the generation and expression of recombinant nucleic acids and their encoded gene products. The detection methods used herein include, for example, cloning and sequencing, ligation of oligonucleotides, use of the polymerase chain reaction and variations thereof (e.g., a PCR that uses 7-deaza GTP), use of single nucleotide primer-guided extension assays, hybridization techniques using target-specific oligonucleotides that can be shown to preferentially bind to complementary sequences under given stringency conditions, and sandwich hybridization methods.

Sequencing may be carried out with commercially available automated sequencers utilizing labeled primers or terminators, or using sequencing gel-based methods. Sequence analysis is also carried out by methods based on ligation of oligonucleotide sequences which anneal immediately adjacent to each other on a target DNA or RNA molecule (Wu and Wallace, Genomics 4: 560-569 (1989); Landren et al., Proc. Natl. Acad. Sci. 87: 8923-8927 (1990); Barany, F., Proc. Natl. Acad. Sci. 88: 189-193 (1991)). Ligase-mediated covalent attachment occurs only when the oligonucleotides are correctly base-paired. The Ligase Chain Reaction (LCR), which utilizes the thermostable Taq ligase for target amplification, is particularly useful for interrogating late onset diabetes mutation loci. The elevated reaction temperatures permit the ligation reaction to be conducted with high stringency (Barany, F., PCR Methods and Applications 1: 5-16 (1991)).

Hybridization reactions may be carried out in a filter-based format, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, bead-based, silicon chip-based and microtiter well-based hybridization formats.

The cloning and expression vectors described herein are introduced into cells or tissues by any one of a variety of known methods within the art. Such methods are described for example in Sambrook et al., Molecular Cloning: A Laboratory Manual, Cold Spring Harbor Laboratory, New York (1992), which is hereby incorporated by reference. See, also, Ausubel et al., Current Protocols in Molecular Biology, John Wiley and Sons, Baltimore, Md. (1989); Hitt et al, “Construction and propagation of human adenovirus vectors,” in Cell Biology: A Laboratory Handbook, Ed. J. E. Celis., Academic Press. 2^{nd} Edition, Volume 1, pp: 500-512, 1998; Hitt et al, “Techniques for human adenovirus vector construction and characterization,” in Methods in Molecular Genetics, Ed. K. W. Adolph, Academic Press, Orlando, Fla., Volume 7B, pp:12-30, 1995; Hitt, et al., “Construction and propagation of human adenovirus vectors,” in Cell Biology: A Laboratory Handbook,“Ed. J. E. Celis. Academic Press. pp:479-490, 1994, also hereby incorporated by reference. The methods include, for example, stable or transient transfection, lipofection, electroporation and infection with recombinant viral vectors.

The protein products of recombined and unrecombined coding sequences may be analyzed using immune techniques. For example, a protein, or a fragment thereof is injected into a host animal along with an adjuvant so as to generate an immune response. Immunoglobulins which bind the recombinant fragment are harvested as an antiserum, and are optionally further purified by affinity chromatography or other means. Additionally, spleen cells may be harvested from an immunized mouse host and fused to myeloma cells to produce a bank of antibody-secreting hybridoma cells. The bank of hybridomas is screened for clones that secrete immunoglobulins which bind to the variant polypeptides but poorly or not at all to wild-type polypeptides are selected, either by pre-absorption with wild-type proteins or by screening of hybridoma cell lines for specific idiotypes that bind the variant, but not wild-type, polypeptides.

Nucleic acid sequences capable of ultimately expressing the desired variant polypeptides are formed from a variety of different polynucleotides (genomic or cDNA, RNA, synthetic olignucleotides, etc.) as well as by a variety of different techniques.

The DNA sequences are expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors contain selection markers (e.g., markers based on tetracycline resistance or hygromycin resistance) to permit detection and/or selection of those cells transformed with the desired DNA sequences. Further details can be found in U.S. Pat. No. 4,704,362.

Polynucleotides encoding a variant polypeptide include sequences that facilitate transcription (expression sequences) and translation of the coding sequences such that the encoded polypeptide product is produced. Construction of such polynucleotides is well known in the art. For example, such polynucleotides include a promoter, a transcription termination site (polyadenylation site in eukaryotic expression hosts), a ribosome binding site, and, optionally, an enhancer for use in eukaryotic expression hosts, and optionally, sequences necessary for replication of a vector.

In addition to microorganisms, mammalian tissue cell culture is used to express and produce the polypeptides of the present invention. Eukaryotic cells are preferred, because a number of suitable host cell lines capable of secreting intact human proteins have been developed in the art, and include the CHO cell lines, various COS cell lines, HeLa cells, myeloma cell lines, Jurkat cells, and so forth. Expression vectors for these cells include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobin genes, SV40, Adenovirus, Bovine Papilloma Virus, Herpes Virus, and so forth. The vectors containing the DNA segments of interest (e.g., polypeptides encoding a variant polypeptide) are transferred into the host cell by well-known methods, which vary depending on the type of cellular host. For example, calcium chloride transfection is commonly utilized for prokaryotic cells, whereas calcium phosphate treatment or electroporation is useful for other cellular hosts.

Adenoviruses with foreign DNA inserted in place of E1 sequences, and optionally also carrying deletions of E3 sequences are conventionally known as “first generation” adenovirus vectors. First generation vectors are of proven utility for many applications. They can be used as research tools for high-efficiency transfer and expression of foreign genes in mammalian cells derived from many tissues and from many species. First generation vectors can be used in development of recombinant viral vaccines when the vectors contain and express antigens derived from pathogenic organisms. The vectors can be used for gene therapy, because of their ability to efficiently transfer and express foreign genes in vivo, and due to their ability to transduce both replicating and nonreplicating cells in many different tissues. Adenovirus vectors are widely used in these applications.

There are many known ways to construct adenovirus vectors. As discussed above, one of the most commonly employed methods is the so-called “two plasmid” technique. In that procedure, two non-infectious bacterial plasmids are constructed with the following properties: each plasmid alone is incapable of generating infectious virus. However, in combination, the plasmids potentially can generate infectious virus, provided the viral sequences contained therein are recombined to constitute a complete infectious virus DNA. According to that method, typically one plasmid is large (approximately 30,000-35,000 nt) and contains most of the viral genome, save for some DNA segment (such as that comprising the packaging signal, or encoding an essential gene) whose deletion renders the plasmid incapable of producing infectious virus. The second plasmid is typically smaller (e.g. 5000-10,000 nt), as small size aids in the manipulation of the plasmid DNA by recombinant DNA techniques. Said second plasmid contains viral DNA sequences that partially overlap with sequences present in the larger plasmid. Together with the viral sequences of the larger plasmid, the sequences of the second plasmid can potentially constitute an infectious viral DNA. Cotransfection of a host cell with the two plasmids produces an infectious virus as a result of recombination between the overlapping viral DNA sequences common to the two plasmids. One particular system in general use by those skilled in the art is based on a series of large plasmids known as pBHG10, pBHG11 and pBHGE3 described by Bett, A. J., Haddara, W., Prevec, L. and Graham, F. L: “An efficient and flexible system for construction of adenovirus vectors with insertions or deletions in early regions 1 and 3,” Proc. Natl. Acad. Sci. US 91: 8802-8806, 1994 and in WO95/00655 (hereby incorporated by reference). Those plasmids contain most of the viral genome and are capable of producing infectious virus but for the deletion of the packaging signal located at the left end of the wild-type viral genome. The second component of that system comprises a series of “shuttle” plasmids that contain the left approximately 340 nt of the Ad genome including the packaging signal, optionally a polycloning site, or optionally an expression cassette, followed by viral sequences from near the right end of E1 to approximately 15 mu or optionally to a point further rightward in the genome. The viral sequences rightward of E1 overlap with sequences in the pBHG plasmids and, via homologous recombination in cotransfected host cells, produce infectious virus. The resulting viruses contain the packaging signal derived from the shuttle plasmid, as well as any sequences, such as a foreign DNA inserted into the polycloning site or expression cassette located in the shuttle plasmid between the packaging signal and the overlap sequences. Because neither plasmid alone has the capability to produce replicating virus, infectious viral vector progeny can only arise as a result of recombination within the cotransfected host cell.

In the Examples which follow, all results are expressed as the means ±1 SEM. Statistical significance was calculated using the Student's t test for comparison of two means or a one way analysis of variance (ANOVA) for the comparison of three or more means. Multiple comparisons were performed using the Neuman Keuls multiple comparison test. P<0.05 was considered significant.

### EXAMPLE 1

### Mice and Cell Lines

Specific pathogen-free, male NIH Swiss mice (6 to 10 weeks old) purchased from the National Cancer Institute (Bethesda, Md.), and 10 week old female FVB/n mice (Taconic Farms, Germantown, N.Y.) were kept in filter topped cages and given ad libitum access to autoclaved food and water. The protocols employed were in direct accordance with the guidelines of the McMaster University Animal Care Committee and the Canadian Council on the Use of Laboratory Animals. The target cell lines for CTL assay, PT0516 and 516MT3 , are kidney fibroblast lines derived from an FVB mouse. The 516MT3 cell line was generated by stably transfecting the PT0516 cells with the polyoma middle T cDNA (PymT).

### EXAMPLE 2

### Recombinant Adenovirus Vectors

The recombinant human type 5 adenoviruses AdCA35 and AdDK1 (hereafter referred to as AdLacZ and AdLuc) contain the β-galactosidase (β-Gal) and firefly luciferase genes, respectively, under the control of the mouse cytomegalovirus (CMV) immediate early promoter and terminated by the SV40 polyadenylation signal inserted into the E1 region of the Ad5 using the BHG10 backbone described by Bett et al., 1994. The recombinant adenovirus vector expressing the polyoma middle T antigen (AdPymT) was previously described by Davison and Hassell 1987. In earlier studies using an established murine cancer model (PymT transgenic mice), we demonstrated that a single injection with the AdPymT vector via different routes (including iv, sc, ip, id and im) can induce PymT specific T cell-mediated immune responses (Wan et al., 1997). For purposes of comparison with the methods and results of this invention, that data is hereby incorporated by reference as if fully set forth herein.

### EXAMPLE 3

### Study Protocol and Establishment of Viral Infection

Mice were anaesthetized with the gaseous anesthetic Enflurane, (Abbott Laboratories, St. Laurent, Quebec) and while unconscious, given an intra-rectal enema of 50% EtOH (v/v) (diluted in dH₂O) using a catheter made of PE50 polyethylene tubing attached to a 1 ml syringe. The catheter was inserted so that the tip was 4 cm proximal to the anus and a total volume of 150 μl was injected. To ensure distribution of the ethanol throughout the colon, mice were held in a vertical position for 30s after the injection. The mice were then left for 3 hours to recover. At this time they were again anaesthetized and for the marker virus studies, NIH Swiss mice were given 5×10⁸ plaque forming units (pfu) of AdLuc or 1×10⁹ pfu AdLacZ virus by enema in a total volume of 100 μl of phosphate buffered saline (PBS), pH 7.4, in an identical fashion to the first enema. Over the next eight days, mice were sacrificed at regular intervals with tissues collected for β-Gal staining or for luciferase quantification. For the immunization studies, the FVB mice were given 1×10⁸ pfu of AdPymT in 100 μl of PBS also by enema. Control (positive for immunization) mice were injected with 1×10⁸ pfu of AdPymT into the footpad.

### EXAMPLE 4

### Histochemical Localization of Beta-Galactosidase (LACZ) Product

Staining for β-Gal expression was as described by Mastrangeli et al., 1993, with minor modifications. Briefly, over the time course of the infection, mice were euthanized, their colons removed, opened longitudinally, flushed of luminal contents and then fixed with 2% formaldehyde in PBS at 4° C. for 1 hour. The intestine was then rinsed twice with PBS and immersed in staining solution containing 5 mM K₄Fe(CN)₆, 5 mM K₃Fe₃(CN)₆, 2 mM MgCl₂, and 0.5 mg/ml of the X-gal stain (5-bromo-4-chloro-3-indolyl-beta-D-galactopyranoside (Boehringer Mannheim Corp., Indianapolis, Ind.) at 37° C. over-night. The stained intestinal tissues were then paraffin-embedded, sectioned at 6 μm, and counterstained with nuclear fast red. Photographs were taken using a Zeiss camera.

### EXAMPLE 5

### Luciferase Detection

To quantify the levels of luciferase produced in vivo, following enema delivery of AdLuc, animals were sacrificed at days 1,2,3,5 and 8 PI, the colon removed, opened longitudinally, cleaned of fecal material, divided in half (proximal vs. distal colon) and kept on ice. Samples of terminal ileum, as well as spleen, liver and mesentery, including the associated lymph nodes were also removed and treated similarly. Tissues were subsequently homogenized in buffer (0.1 M potassium phosphate pH 7.8, 1 mM PMSF, and 10 ug/ml aprotinin) with a tissue homogenizer, and centrifuged to remove debris. The homogenates were then immediately assayed for luciferase activity as previously described (Mittal et al, 1993).

### EXAMPLE 6

### CTL Assay

Popliteal lymphocytes draining the footpad, and iliac lymphocytes, draining the distal colon, were harvested from mice 5 days after immunization with AdPymT, as described (Wan et al., 1997). After 3 days culture (without restimulation), the cells were tested for cytolytic activity against the 516MT3 cells and the parental PT0516 cells which were both labeled with ⁵¹Cr. Release of ⁵¹Cr was determined using a gamma counter, as described (Wan et al., 1997).

### EXAMPLE 7

### Luminal Adenoviral Gene Delivery

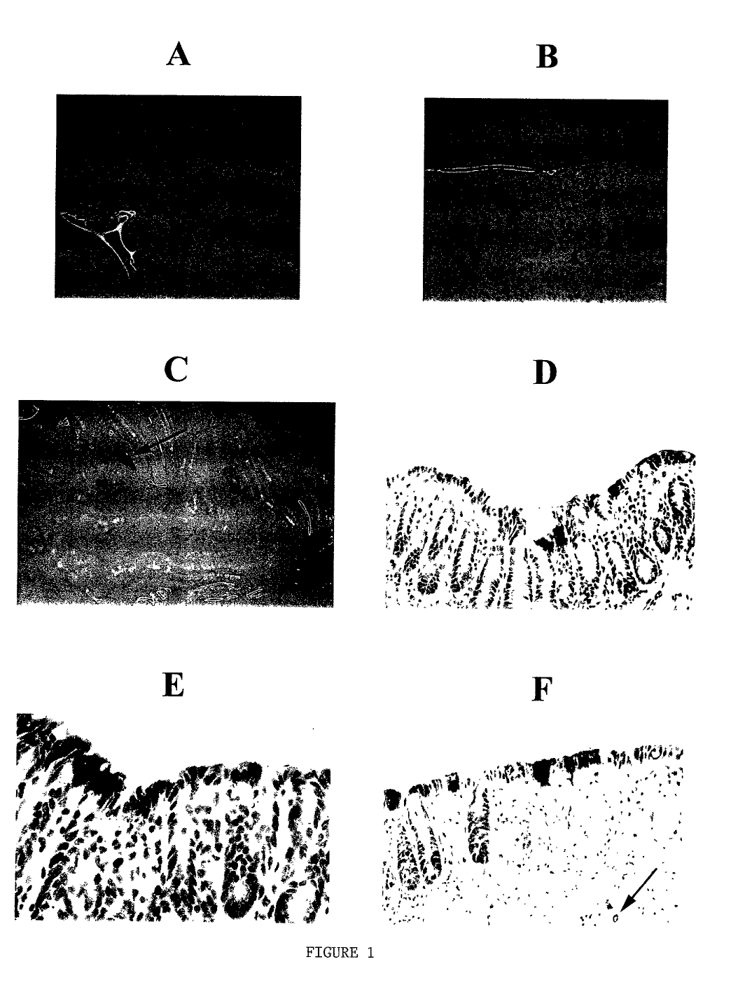

Initial studies were performed to determine the baseline transgene expression obtained within the colon of mice receiving either the AdLacZ or the AdLuc viruses delivered in enema form without any pretreatment of the colon. A few β-Gal positive cells were detected in the distal colon (see FIG. 1A) but not in the proximal colon, and luciferase activity did not rise above background levels (not shown). Pretreating the colon with an enema of the mucolytic agent, dithiothreitol, followed 3 hours later with adenoviral vectors, increased transfection efficiency, however the number of positively staining cells was still not impressive. Ttransiently disrupting both the epithelial and mucus layers of the colon through the intra-rectal instillation of a solution of 50% ethanol resulted in greatly enhanced levels of transgene expression.

### EXAMPLE 8

### Ethanol Pretreatment Study Protocol

Dilute ethanol is known to transiently disrupt mucosal barriers in the stomach (Jacobson, 1986), and more recently has been used as the vehicle and barrier breaker to deliver haptenating agents such as trinitrobenzene sulfonic acid (TNBS) to the colon to induce experimental colitis in mice (Neurath et al, 1995) and rats (Wallace et al., 1989). Since enema delivery of ethanol alone is known to cause only mild irritation (Neurath et al., 1995; Wallace et al., 1989), we examined whether pretreating the colon with dilute ethanol would facilitate adenoviral infection of the colon. Different dilutions of ethanol (30, 40 and 50%) and different time courses of viral delivery following pretreatment were examined, with the best results found using a solution of 50% ethanol, followed 3 hours later by a second enema containing the adenovectors. Surprisingly, no transgene expression was seen if the viruses were given less than 3 hours after the ethanol, while transgene expression decreased if the viruses were given more than 3 hours after pretreatment.

### EXAMPLE 9

### Beta-Galactosidase Staining and Histology

After the colon was opened longitudinally, stained for β-gal, and examined under a dissecting microscope, numerous small blue stained cells were seen on the mucosal surface (see FIG. 1B), but none were detected on the serosal surface, nor in the mesentery. β-gal positive cells were found throughout the colon, although greater numbers were observed in the distal region. Strong staining of dome-like areas in the proximal colon (FIG. 1C) likely indicates the infection of M cells overlying lymphoid follicles of the colon, as M cells were recently shown to be particularly susceptible to adenoviral infection (Foreman et al., 1998). No staining was seen in the caecum or small bowel. When a time course was performed, similar large numbers of positively staining cells were seen at days 1 and 2, while the number of stained cells decreased by approximately 50% by day 3 . By day 5, a scattering of blue cells was still evident, and by day 8 all evidence of β-gal transgene expression was gone.

Examination of cross sections of the infected colon identified the majority of the infected cells as intestinal epithelial cells (IEC) (see FIGS. 1D & E), with staining occasionally reaching down into the crypts (not shown). Up to 10% of epithelial cells in these cross sections were β-gal positive. Infrequently, scattered cells in the subepithelial layers were also stained (FIG. 1F), however the identity of these lamina propria cells is unknown. Little histological damage or signs of inflammation were noted in tissues removed from mice given ethanol alone. Occasional signs of mild inflammation, and some enlarged lymphocytic aggregates were noted in tissues removed from mice given ethanol followed by adenoviral enemas, likely reflecting the host's response against the viral vectors.

### EXAMPLE 11

### Luciferase Expression

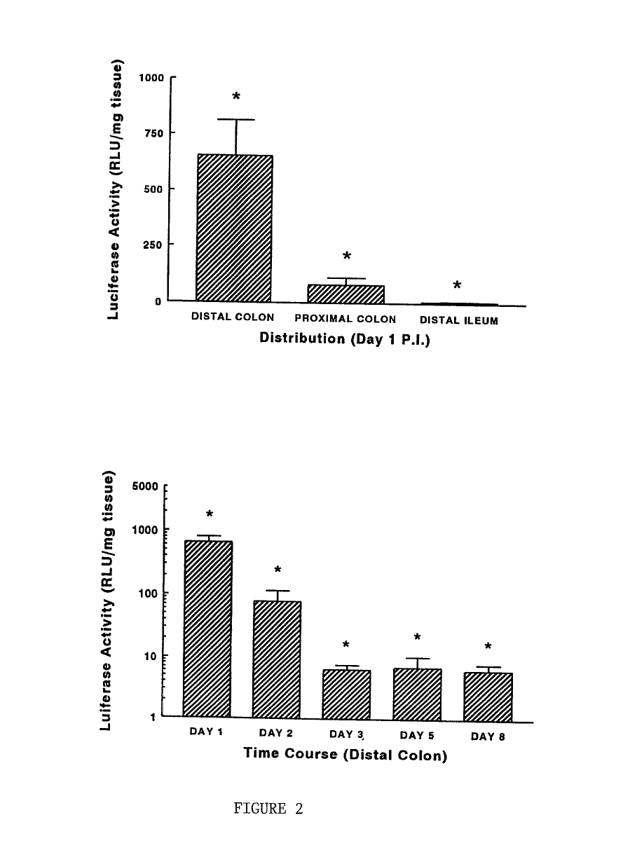

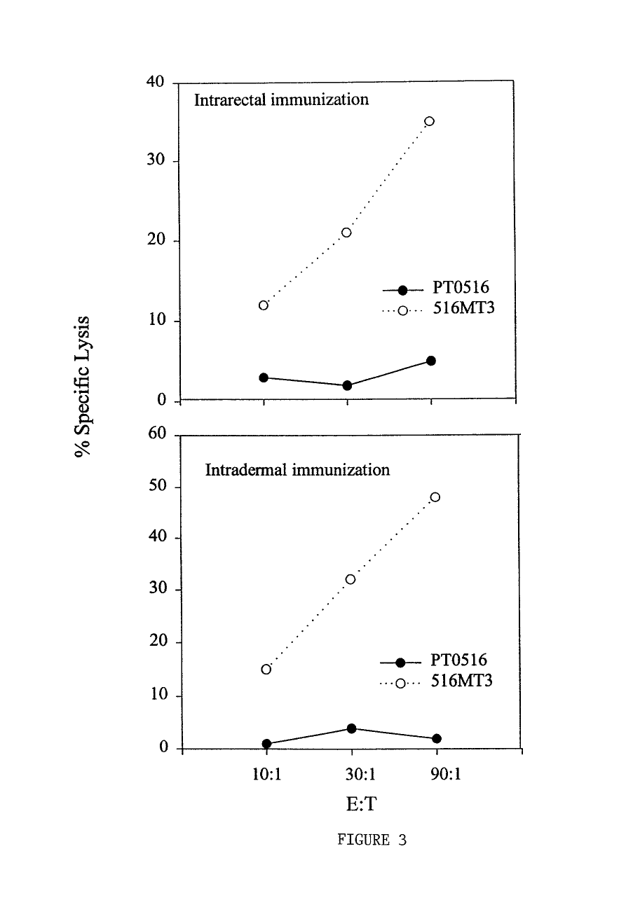

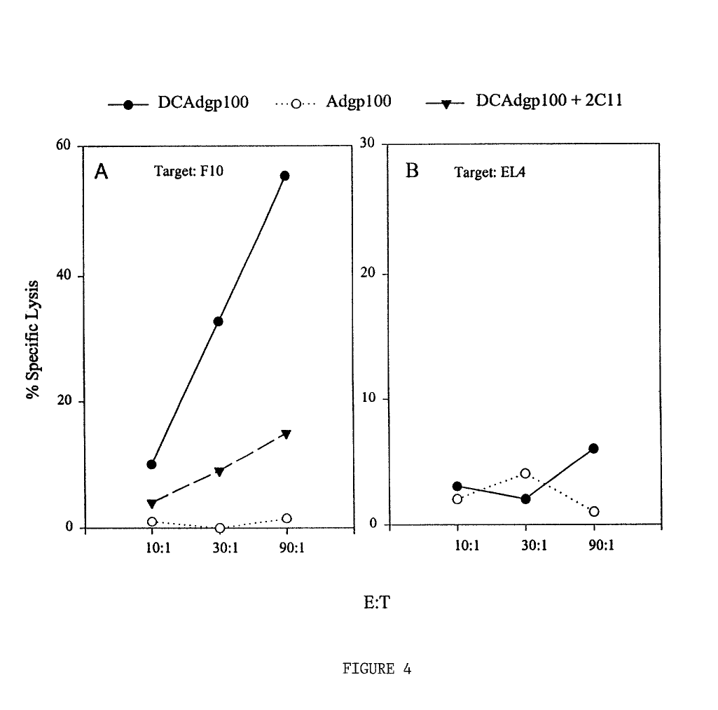

While β-Gal staining identifies infected cells expressing the LacZ transgene, it is not a reliable reporter gene for quantifying transgene expression. Therefore, we also examined luciferase reporter expression. Similar to our observations with the β-Gal staining, expression was found predominantly in the distal colon, although limited expression was also detected in the proximal colon (see FIG. 2A). Little expression was detected in the ileum, and no expression was detected in the spleen, liver, mesenteric or iliac lymph nodes, indicating that the infection and transgene expression was selective to the GI tract. As shown in FIG. 2B, luciferase activity was strongest at day 1, with luciferase activity of 650 relative light units (RLU)/mg tissue, equivalent to approximately 2-3 ng of luciferase protein in the colon. Expression was reduced at day 2 and while the levels of luciferase detected at days 3, 5 and 8 PI were further reduced, they were still significantly elevated over baseline activity (<1RLU/mg tissue).

### EXAMPLE 12

### CTL Immune Response

Since applications proposed for intestinal gene therapy include vaccination and will require the development of an immune response against transgenic proteins, we examined the efficacy of adenoviral gene transfer to the colon in inducing a cytotoxic immune response against a well-characterized tumor antigen, PymT. Mice are given an intra-rectal enema of 50% ethanol, 150 microliters through a catheter made of PE50 polyethelene tubing. The fluid is administered within the area 4 cm proximal to the anus. After 3 hr, the mice are anaesthetised and 5×10⁸ pfu of purified vector is administered in 100 microliters of saline in an identical manner to the first ethanol administration. When an Adenovirus vector encoding the tumour antigen PymT is used, after 8 days cells are removed from the ileac lymph nodes and are shown to be primed for Cytotoxic T cell activity specific for PymT antigen, as determined in a standard CTL assay against PymT expressing cell targets. As seen in FIG. 3, intrarectal immunization using the AdPymT virus led to the strong induction of cytotoxic T cells in the iliac nodes draining the colon which specifically lysed a cell line expressing the PymT antigen, but not the parental cell line, in a dose dependent manner. CTL efficacy induced by intra-rectal immunization was of a similar magnitude to that found in popliteal lymphocytes generated by intradermal footpad injection of the AdPymT vector.

### EXAMPLE 13

### Methods of Intestinal Delivery of Nucleic Acids in Animals and Humans

Naturally, based on the present disclosure, those skilled in the art will be enabled to develop variations on the nucleic acid delivery protocols disclosed herein. It will be appreciated that the methods employed herein for gene presentation to murine intestinal cells is not directly applicable to gene presentation to human intestinal cells. However, those skilled in the art will appreciate that suppositories comprising nucleic acids, muco-disruptive agents, or both in combination may be employed by humans or in large animals of agricultural significance. Enema, injection, instillation, sprays and the like may all be employed for this purpose. Thus, in humans, a suppository comprising agents that melt at body temperature to release mucolytic or muco-disruptive agents, nucleic acids or both is employed with convenience for the purpose of delivering adenoviral vectors, retroviral vectors, naked nucleic acid constructs and the like in order to treat or prevent a wide variety of pathologic conditions.

### References

1. Jobin C, Panja A, Hellerbrand C, Iimuro Y, Didonato J, Brenner D A, Sartor R B. Inhibition of proinflammatory molecule production by adenovirus-mediated expression of a nuclear factor kB super-repressor in human intestinal epithelial cells. J. Immunol 1998; 160:410-418.

2. Cheng D Y, Kolls J K, Lei D, Noel R A. In vivo and in vitro gene transfer and expression in rat intestinal epithelial cells by E1 -deleted adenoviral vector. Hum. Gene Ther. 1997; 8:755-764.

3. Baca-Estrada M E, Liang X, Babiuk L A, Yoo D. Induction of mucosal immunity in cotton rats to haemagglutinin-esterase glycoprotein of bovine coronavirus by recombinant adenovirus. Immunology 1995; 86:134-140.

4. Addison C L, Braciak T, Ralston R, Muller W J, Gauldie J, Graham F L. Intratumoral injection of an adenovirus expressing interleukin 2 induces regression and immunity in a murine breast cancer model. Proc Natl Acad USA 1995; 92:8522-8526.

5. Hogaboam C M, Valiance B A, Kumar A, Addison C L, Graham F L, Gauldie J, Collins S M. Therapeutic effects of interleukin-4 gene transfer in experimental inflammatory bowel disease. J Clin Invest 1997; 100:2766-2776.

6. Sandberg J W, Lau C, Jacomino M, Finegold M, Henning S J. Improving access to intestinal stem cells as a step toward intestinal gene transfer. Hum Gene Ther 1994; 5:323-329.

7. Sferra T J, McNeely D, Johnson P R. Gene transfer to the intestinal tract: A new selective injection of the superior mesenteric artery. Hum Gene Ther 1997; 8:681-687.

8. Foreman P K, Wainwright M J, Alicke B, Kovesdi, Wickham T J, Smith J G, Meier-Davis S, Fix J A, Daddona P, Gardner P, Huang M T F. Adenovirus-mediated transduction of intestinal cells in vivo. Human Gene Therapy 1998, 9, 1313-1321.

9. Brown G R, Thiele D L, Silva M, Beutler B. Adenoviral vectors given intravenously to immunocompromised mice yield stable transduction of the colonic epithelium. Gastroenterology. 1997; 122:1586-1594.

10. Bett A J, Haddara W, Prevec L, Graham F L. An efficient and flexible system for construction of adenovirus vectors with insertions or deletions in early regions 1 and 3 . Proc Natl Acad Sci USA, 1994, 91:8802-8806.

11. Davidson D, Hassell J A. Overproduction of polyomavirus middle T antigen in mammalian cells through the use of an adenovirus vector. J. Virol. 1987, 61:1226-1239.

12. Wan Y, Bramson J, Carter R, Graham F L, Gauldie J. Dendritic cells transduced with an adenoviral vector encoding a model tumor-associated antigen for tumor vaccination. Hum Gene Ther. 1997, 8:1355-1363.

13. Mastrangeli A, Danel C, Rosenfeld M A, Stratford-Perricaudet L, Perricaudet M, Pavirani A, Lecocq J P, Crystal R G. Diversity of airway epithelial cell targets for in vivo recombinant adenovirus-mediated gene transfer. J Clin Invest 1993; 91:225-234.

14. Mittal S K, McDermott M R, Johnson D C, Prevec L, Graham F L. Monitoring foreign gene expression by a human adenovirus-based vector using the firefly luciferase gene as a reporter. Virus Research 1993; 28:67-90.

15. Neurath M F, Fuss I, Kelsall B L, Stuber E, Strober W. Antibodies to interleukin 12 abrogates established experimental colitis in mice. J. Exp. Med. 1995; 182:1281-1290.

16. Wallace J L, MacNaughton W K, Morris G P, Beck P L. Inhibition of leukotriene synthesis markedly accelerates healing in a rat model of inflammatory bowel disease. Gastroenterology 1989; 96:29-36.

17. Jacobson E. D. Direct and adaptive cytoprotection. Dig.Dis. Sci. 1986; 31:28S-31S.

18. Mayer L. The role of the epithelium in mucosal immunity. Res. Immunol. 1997; 148:498-504.

19. Lozier J N, Yankaskas J R, Ramsey W J, Chen L, Berschneider H, Morgan R A. Gut epithelial cells as targets for gene therapy of haemophilia. Hum. Gene Ther. 1997; 8:14811-14890.

20. Lipkin M, Sherlock P, and Bell B. Cell proliferation kinetics in the gastrointestinal tract of man. II. Cell renewal in stomach, ileum, colon, and rectum. Gastroenterology 1963; 45:721-729.

21. Yei S, Mittereder N, Wert S, Whitsett J A, Wilmott R W, Trapnell B C. In vivo evaluation of the safety of adenovirus-mediated transfer of the human cystic fibrosis transmembrane conductance regulator cDNA to the lung. Hum. Gene. Ther. 1994; 5:731-744.

22. Rolph M. S., and Ramshaw I. A. Recombinant viruses as vaccines and immunological tools. Curr Opin Immunol 1997; 9:517-524.

23. Parkin D. M., Pisani P., and Ferlay J. Estimates of the worldwide incidence of 25 major cancers in 1990. Int. J. Cancer. 1999; 80:827-841.

24. French G. L.. Enterococci and vancomycin resistance. Clin Infect Dis 1998 Suppl. 1:S75-S83.

25. Wilson J M. Gene therapy for cystic fibrosis: challenges and future directions. J. Clin. Invest. 1995; 96:2547-2554.

26. MacDonald T T. Viral vectors expressing immunoregulatory cytokines to treat inflammatory bowel disease. Gut 1998; 42:460-461.

27. Shibata, H., et al., Rapid Colorectal Adenoma Formation Initiated by Conditional Targeting of the Apc Gene. Science 1997; 278:120-123.

28. Wirtz, et al., Efficient Gene Delivery to the Inflamed Colon by Local Administration of Recombinant Adenoviruses with Normal or Modified Fibre Structure. Gut 1999; 44:800-807.

What is claimed is:

1. A method for delivery of a pharmaceutical composition to gastrointestinal cells in a recipient in need thereof which comprises:
(a) contacting the intended site of delivery of said pharmaceutical composition with an agent adequate to cause a temporary disruption of the mucosal lining covering said gastrointestinal cells; and
(b) concurrently or subsequent to said contacting of step (a), contacting said gastrointestinal or genitourinary cells with said pharmaceutical composition, wherein said pharmaceutical composition comprises a nucleic acid or a cell comprising a nucleic acid, the expression of which is desired in said gastrointestinal cells.

2. The method according to claim 1 wherein said pharmaceutical composition further comprises a protein, an antibiotic, an anti-inflammatory, an analgesic, an anti-neoplastic, a cell, or a mixture thereof.

3. The method according to claim 2 wherein said pharmaceutical composition comprises a nucleic acid or a cell comprising a nucleic acid encoding (i) an RNA product which is the antisense of a gene product, the expression of which is intended to be suppressed in said gastrointestinal cells or (ii) a peptide or protein the expression of which is desired in said gastrointestinal cells.

4. The method according to claim 3 wherein said protein is a biologically active protein capable of effecting desired biological functions or is an immunogenic protein against which immune responses are intended to be induced.

5. The method according to claim 4 wherein said protein is selected from the group consisting of a tumor antigen, a cytokine, a growth factor, a marker gene product, an enzyme, a receptor, a receptor antagonist, and a structural protein.

6. The method according to claim 5 wherein said tumor antigen is the PymT antigen, wherein said growth factor or cytokine is an interleukin or is a tissue growth factor, and wherein said receptor antagonist is an interleukin receptor or growth factor antagonist.

7. The method according to claim 3 wherein said nucleic acid comprises sufficient gene regulatory control sequences to achieve efficient expression of encoded sequences upon uptake of said nucleic acid by said gastrointestinal or genitourinary cells.

8. The method according to claim 4 wherein said nucleic acid comprises viral sequences.

9. The method according to claim 8 wherein said viral sequences are selected from adenoviral sequences and retroviral sequences.

10. The method according to claim 9 wherein said adenoviral sequences are insufficient to encode a replication-competent virus in the absence of adenoviral sequences or functions provided in trans.

11. The method according to claim 1 wherein said agent adequate to cause a temporary disruption of the mucosal lining covering said gastrointestinal or genitourinary cells is a mucolytic agent, a mucodistruptive agent, a penetration enhancing agent, or a combination of such agents.

12. The method according to claim 11 wherein said agent is administered by means of a spray, suppository, or enema.

13. The method according to claim 12 wherein said agent is selected from the group consisting of a non-toxic alcohol, DMSO, a mucolytic enzyme, N-acetyl cysteine, and combinations thereof.

14. The method according to claim 13 wherein said alcohol is ethyl alcohol.

15. The method according to claim 14 wherein said ethyl alcohol comprises about a 5 to 75% concentration of ethyl alcohol.

16. The method according to claim 15 wherein said ethyl alcohol comprises about a 25 to 60% concentration of ethyl alcohol.

17. The method according to claim 15 wherein said ethyl alcohol comprises about a 50% concentration of ethyl alcohol.

18. The method according to claim 1 which comprises intrarectal administration of about a 50% solution of ethyl alcohol about three hours prior to administration of a nucleic acid encoding a gene product the expression of which in intestinal epithelial and other intestinal cells is desired.

19. The method according to claim 18 wherein said nucleic acid encodes a gene product selected from the group consisting of tumor antigen, a growth factor, a cytokine, a receptor, a receptor antagonist, a structural protein, an antisense nucleic acid, an antigen encoded by a pathogen against which immune responses are desired to be elicited, and combinations thereof.

20. A method for inducing extended transgene expression in the intestine which comprises simultaneous treatment or pre-treatment of the intestinal tract with a mucous membrane disruptive agent and contacting the thus treated intestinal tract with a nucleic acid.

21. The method of claim 20 wherein said nucleic acid comprises a biologically active gene.

22. The method according to claim 21 wherein said nucleic acid is contained within a vector or a cell.

23. The method according to claim 20 wherein said nucleic acid is in a precipitated or encapsulated state, such that nucleic acid is released for uptake by intestinal cells over an extended period of time.

24. The method according to claim 20 wherein said method is repeated.

25. A composition comprising a mucolytic agent or a mucodisruptive agent in combination with a biologically active nucleic acid.

26. A suppository comprising a biologically active nucleic acid.

27. A method for treating or preventing a pathologic condition which comprises temporary disruption of the mucosal lining of the intestine and contact of the thus treated intestine with a biologically active nucleic acid.

28. The method according to claim 27 for prevention or treatment of intestinal tumors, treatment or prevention of sexually transmitted diseases, or treatment or prevention of inflammatory bowel disease.
